(19) United States
(12) Patent Application Publication
(10) Pub. No.: US 2024/0378741 A1
(43) Pub. Date: Nov. 14, 2024

(54) METHOD AND APPARATUS FOR DETECTING AND DETERMINING THE LOCATION OF OBJECTS OF INTEREST IN AN IMAGE

(71) Applicant: 3D SMILE USA, INC., New York, NY (US)

(72) Inventors: Marina DOMRACHEVA, New York, NY (US); Ivan PISCHASOV, Panama City (PA)

(73) Assignee: 3D SMILE USA, INC., New York, NY (US)

(21) Appl. No.: 18/316,319

(22) Filed: May 12, 2023

(51) Int. Cl. G06T 7/70; A61C 7/00; A61C 13/34; G06T 7/11; G06V 10/25; G06V 10/26; G06V 10/82; G16H 30/40; G16H 50/50

(52) U.S. Cl. CPC G06T 7/70; A61C 7/002; A61C 13/34; G06T 7/11; G06V 10/25; G06V 10/26; G06V 10/82; G16H 30/40; G16H 50/50; G06T 2207/10081; G06T 2207/20021; G06T 2207/20076; G06T 2207/20081; G06T 2207/20084; G06T 2207/30036; G06T 2207/30201; G06V 2201/03

(57) ABSTRACT

The present disclosure relates to an apparatus, comprising: processing circuitry configured to obtain an image, apply a first model and a second model to the obtained image, determine, based on the first model, preliminary locations of objects in the obtained image, determine, based on the second model, adhesion locations disposed between the objects in the obtained image, and determine, based on the preliminary locations and the adhesion locations, refined locations of the objects in the obtained image.

### BACKGROUND

### Field of the Disclosure

The present disclosure relates to a dental image processing method for teeth instance segmentation from an obtained image with fine-grained boundaries.

### Description of the Related Art

Orthodontics, generally, and dental alignment, in particular, is a well-developed area of dental care. For patients with misaligned teeth, traditional braces or, more recently, clear aligners, offer a strategy for improved dental function and aesthetics though gradual teeth movements. These gradual teeth movements slowly move a tooth until a desired final position is reached. Notably, both 2D and 3D images and models can be used to help visualize teeth, jaws, and other important features in a dental region of interest.

3D models of the teeth and bone can be used to great effect in a correction treatment plan. The models provide a more comprehensive and accurate representation of a patient's dental and craniofacial anatomy. The models allow technicians or orthodontists to visualize an entire treatment area in three dimensions, providing a better understanding of the interplay between the teeth, jaws, and surrounding structures. With the 3D models, technicians or orthodontists can simulate various treatment scenarios and predict an outcome of a given procedure. This allows them to create an effective correction treatment plan that addresses a patient's specific needs, while minimizing risk of potential complications. Thus, improving accuracy of teeth detection and location remains an area of interest.

The foregoing “Background” description is for the purpose of generally presenting the context of the disclosure. Work of the inventors, to the extent it is described in this background section, as well as aspects of the description which may not otherwise qualify as prior art at the time of filing, are neither expressly nor impliedly admitted as prior art against the present invention.

### SUMMARY

The present disclosure relates to an apparatus, including processing circuitry configured to obtain an image, apply a first model and a second model to the obtained image, determine, based on the first model, preliminary locations of objects in the obtained image, determine, based on the second model, adhesion locations disposed between the objects in the obtained image, and determine, based on the preliminary locations and the adhesion locations, refined locations of the objects in the obtained image.

The present disclosure additionally relates to a method, including obtaining an image, applying a first model and a second model to the obtained image, determining, based on the first model, preliminary locations of objects in the obtained image, determining, based on the second model, adhesion locations disposed between the objects in the obtained image, and determining, based on the preliminary locations and the adhesion locations, refined locations of the objects in the obtained image.

The present disclosure additionally relates to a non-transitory computer-readable storage medium storing computer-readable instructions that, when executed by a computer, cause the computer to perform a method of pixel-based classification of medical images, the method including obtaining an image, applying a first model and a second model to the obtained image, determining, based on the first model, preliminary locations of objects in the obtained image, determining, based on the second model, adhesion locations disposed between the objects in the obtained image, and determining, based on the preliminary locations and the adhesion locations, refined locations of the objects in the obtained image.

The foregoing paragraphs have been provided by way of general introduction and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

The patent or application file contains at least one drawing executed in color. Copies of this patent or patent application publication with color drawing(s) will be provided by the office upon request and payment of the necessary fec.

A more complete appreciation of the disclosure and many of the attendant advantages thereof will be readily obtained as the same becomes better understood by reference to the following detailed description when considered in connection with the accompanying drawings, wherein:

FIG. 1 is a flowchart for a method of processing a dental image, according to an embodiment of the present disclosure.

FIG. 2A is a volumetric dental image of a jaw and skull, according to an embodiment of the present disclosure.

FIG. 2B is a 2D image of a jaw and teeth based on a computed tomography (CT) scan, according to an embodiment of the present disclosure.

FIG. 2C is a 2D image of a jaw and teeth based on a CT scan, according to an embodiment of the present disclosure.

FIG. 3 is an illustration of a jaw region of interest (ROI) in a volumetric image, according to an embodiment of the present disclosure.

FIG. 4A is an illustration of a jaw ROI in a 2D image, according to an embodiment of the present disclosure.

FIG. 4B is an illustration of a jaw ROI in a 2D image, according to an embodiment of the present disclosure.

FIG. 5 is an illustration of a sliding window inference process, according to an embodiment of the present disclosure.

FIG. 6A is an illustration of a training image, according to an embodiment of the present disclosure.

FIG. 6B is an illustration of a training image, according to an embodiment of the present disclosure.

FIG. 7A is an illustration of an output for a trained model, according to an embodiment of the present disclosure.

FIG. 7B is an illustration of an output for a trained model, according to an embodiment of the present disclosure.

FIG. 8A is an illustration of the segmentation issue in a 3D rendering, according to an embodiment of the present disclosure.

FIG. 8B is an illustration of a training image, according to an embodiment of the present disclosure.

FIG. 8C is an illustration of a training image, according to an embodiment of the present disclosure.

FIG. 9A is an illustration of an output for a trained model, according to an embodiment of the present disclosure.

FIG. 9B is an illustration of an output for a trained model, according to an embodiment of the present disclosure.

FIG. 10A is an illustration of an input for a teeth separation process, according to an embodiment of the present disclosure.

FIG. 10B is an illustration of an input for a teeth separation process, according to an embodiment of the present disclosure.

FIG. 11 is an illustration of an input for a teeth separation process, according to an embodiment of the present disclosure.

FIG. 12A is an illustration of an input for a teeth separation process, according to an embodiment of the present disclosure.

FIG. 12B is an illustration of an input for a teeth separation process, according to an embodiment of the present disclosure.

FIG. 13A is an illustration of an output for a teeth separation process, according to an embodiment of the present disclosure.

FIG. 13B is an illustration of an output for a teeth separation process, according to an embodiment of the present disclosure.

FIG. 14A is an illustration of an input for a teeth separation process, according to an embodiment of the present disclosure.

FIG. 14B is an illustration of an input for a teeth separation process, according to an embodiment of the present disclosure.

FIG. 15 is an illustration of an input for a teeth separation process, according to an embodiment of the present disclosure.

FIG. 16A is an illustration of an input for a teeth separation process, according to an embodiment of the present disclosure.

FIG. 16B is an illustration of an input for a teeth separation process, according to an embodiment of the present disclosure.

FIG. 17A is an illustration of an output for a teeth separation process, according to an embodiment of the present disclosure.

FIG. 17B is an illustration of an output for a teeth separation process, according to an embodiment of the present disclosure.

FIG. 18A is an illustration of an output for a teeth splitting process, according to an embodiment of the present disclosure.

FIG. 18B is an illustration of an output for a teeth splitting process, according to an embodiment of the present disclosure.

FIG. 19A shows an example of a general artificial neural network (ANN) having N inputs, K hidden layers, and three outputs.

FIG. 19B shows a non-limiting example in which the DL network is a convolutional neural network (CNN).

FIG. 20 is a schematic of exemplary hardware for implementation of a dental image processing protocol, according to an embodiment of the present disclosure.

### DETAILED DESCRIPTION

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). Reference throughout this document to “one embodiment”, “certain embodiments”, “an embodiment”, “an implementation”, “an example” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

Currently, orthodontist and dental technicians develop teeth movement plans based upon initial and ideal final tooth positions. Having high accuracy when developing the teeth movement plans can lead to better final results. Inaccurate models can lead to incorrect diagnoses, which can result in ineffective treatments that either fail to address the patient's needs or cause additional problems. For example, if the 3D model of a patient's teeth is inaccurate, orthodontic aligners manufactured for that patient's teeth using the model may not fit properly, leading to discomfort and a longer treatment time.

Moreover, accurate 3D models also help to minimize the need for repeat imaging and re-treatment, which can save time, money, and resources. By having a clear and detailed understanding of the patient's anatomy, the dentist or orthodontist can plan the most effective treatment that addresses the patient's specific needs, while minimizing the risk of potential complications.

As such, the present disclosure describes an orthodontic treatment approach that achieves high accuracy detection and location determination of teeth in obtained images.

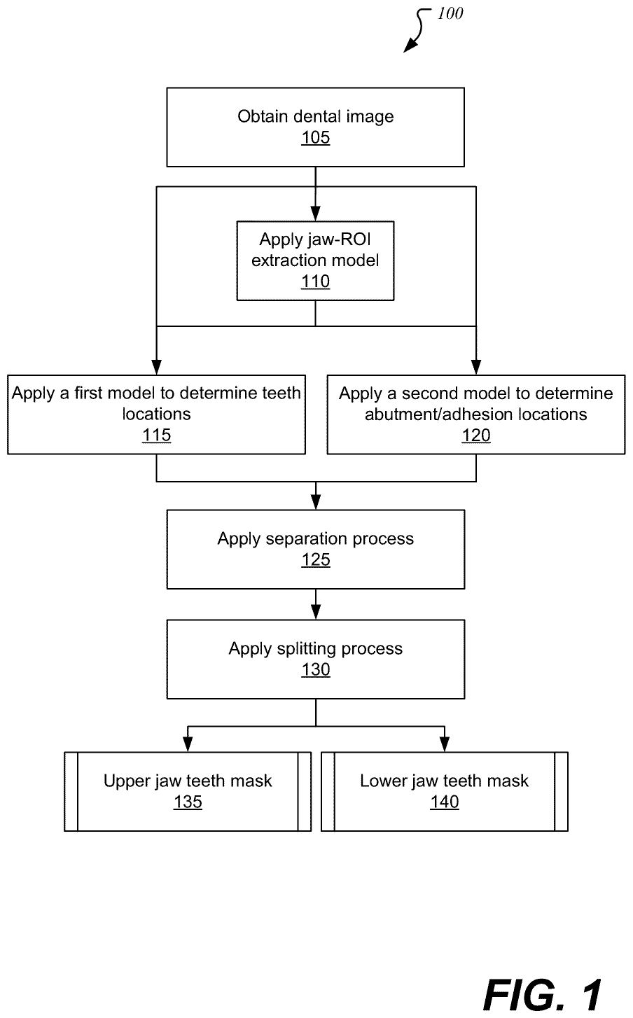

FIG. 1 is a flowchart for a method 100 of processing a dental image, according to an embodiment of the present disclosure. A brief overview of the method 100 is described herein, followed by a more detailed explanation of the steps with reference to the figures.

In an embodiment, at step 105, a dental image is obtained. For example, the dental image can be a computed tomography (CT) image.

In an embodiment, at optional step 110, a jaw region of interest (ROI) model can be applied to the obtained dental image to determine a subset of the dental image to analyze. This can reduce processing power needed to process the dental image in later steps. As described below, processing the entire dental image will need more processing resources compared to processing a subset of the entire dental image, wherein the subset comprises the jaw ROI. The jaw ROI can include, for example, teeth, an upper jaw area, and a lower jaw area.

In an embodiment, at step 115, a first model can be applied to the dental image to determine locations of teeth in the dental image. For example, the first model can be a segmentation model to segment the teeth.

In an embodiment, at step 120, a second model can be applied to the dental image to determine abutment or adhesion or collision locations between the teeth. For example, the second model can be a segmentation model to determine the separation areas between the teeth.

In an embodiment, at step 125, a separation process can be applied to an output of the first model and an output of the second model to further refine the output of the first model. The separation process can further separate or segment the teeth identified in the dental image.

In an embodiment, at step 130, a splitting or grouping process can be applied to split the teeth between an upper jaw and a lower jaw to generate an upper jaw teeth binary mask 135 and a lower jaw teeth binary mask 140.

A segmentation process (or model) can be a computer-based process used to identify and distinguish one or more objects from a given image. The goal of the segmentation process can be to divide the image into distinct regions or segments so that each region can be identified and labeled separately. In the context of a CT image of teeth as the dental image, the segmentation process can be used to identify and locate each individual tooth. The segmentation process can begin by analyzing the CT image and assigning each pixel (or voxel) a numerical value. This is known as image segmentation. The numerical values of the pixels can be used to compare and contrast the different features in the CT image, such as color, material, shape, etc. The process can then use this information to separate the regions in the CT image based on similarity or contrast.

In an embodiment, each tooth can be identified and located via, for example, a sliding window segmentation model. This is done by analyzing each region and looking for features that are characteristic of teeth. For example, the process can look for shapes that resemble the shape of a tooth, or for areas of high contrast between a gum line and a tooth surface. Once a tooth has been identified, the process can then use the tooth location to identify the other teeth in the image. This can be performed by, for example, looking at the spatial relationships between the teeth and making sure the teeth are in the correct order. The process can also use the location of the teeth to measure teeth size and shape. This can provide information about the bite and the alignment of the teeth.

Once the segmentation process has identified and located each tooth, a 3D model of the teeth and jaw can then be generated based on a mask that is formed based on the identified and located teeth. The 3D model can be used to accurately measure the size and shape of the teeth and can also be used to create a virtual dental impression. This impression can then be used to create a 3D printed model of the teeth and jaw, which can be used for further analysis and treatment. Notably, the segmentation process can include various sub-processes and the use of a neural network to further refine and improve the accuracy of the generated masks. Additional details are described herein, such as the dental images, the input training datasets, the resulting outputs, and the improved accuracy of the refined processes.

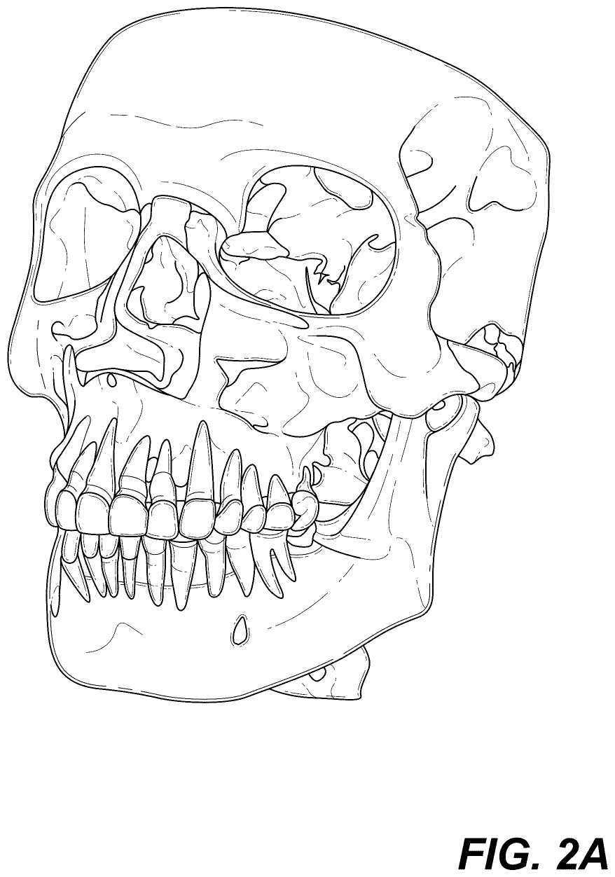

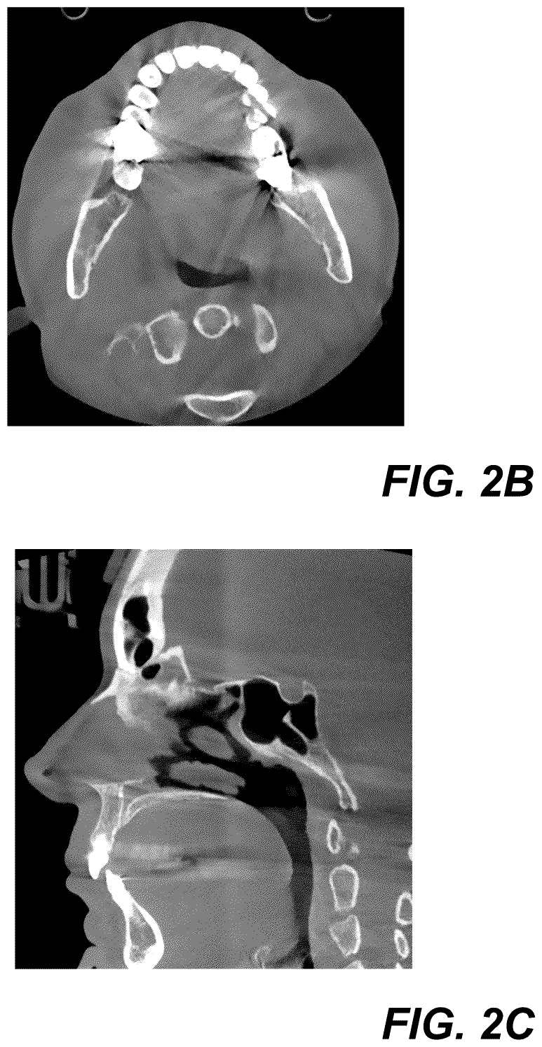

To this end, FIG. 2A is a volumetric dental image of a jaw and skull, according to an embodiment of the present disclosure. In an embodiment, and with reference to step 105 of FIG. 1, the dental image can be a CT image rendered as a volumetric image based on obtained image data from a CT system. CT systems and methods are widely used, particularly for medical imaging and diagnosis. CT systems generally create images of one or more sectional slices through a subject's body. A radiation source, such as an X-ray source, irradiates the body from one side. At least one detector on the opposite side of the body receives radiation transmitted through the body. The attenuation of the radiation that has passed through the body is measured by processing electrical signals received from the detector.

A CT sinogram indicates attenuation through the body as a function of position along a detector array and as a function of the projection angle between the X-ray source and the detector array for various projection measurements. In a sinogram, the spatial dimensions refer to the position along the array of X-ray detectors. The time/angle dimension refers to the projection angle of X-rays, which changes as a function of time during a CT scan. The attenuation resulting from a portion of the imaged object (e.g., a vertebra) will trace out a sine wave around the vertical axis. Those portions farther from the axis of rotation correspond to sine waves with larger amplitudes, and the phase of the sine waves corresponds to the angular positions of objects around the rotation axis. Performing an inverse Radon transform—or any other image reconstruction method-reconstructs an image from the projection data in the sinogram.

To this end, FIG. 2B is a 2D image of a jaw and teeth based on a CT scan, according to an embodiment of the present disclosure. Similarly, FIG. 2C is a 2D image of a jaw and teeth based on a CT scan, according to an embodiment of the present disclosure. In an embodiment, the CT images in FIGS. 2B and 2C are image reconstructions based on the projection data of a sinogram obtained during the CT scan. Each reconstructed image provides a 2D view of the patient or object at a particular time and location. In FIG. 2B, the 2D view is along a plane perpendicular to an axis of the patient to show all the teeth in one jaw at once, where the axis runs from head to toe of the patient. In FIG. 2C, the 2D view is along a plane parallel to an axis of the patient to show both jaws at once. The 2D reconstructed views and corresponding sinograms can be used to generate the 3D volumetric rendering shown in FIG. 2A.

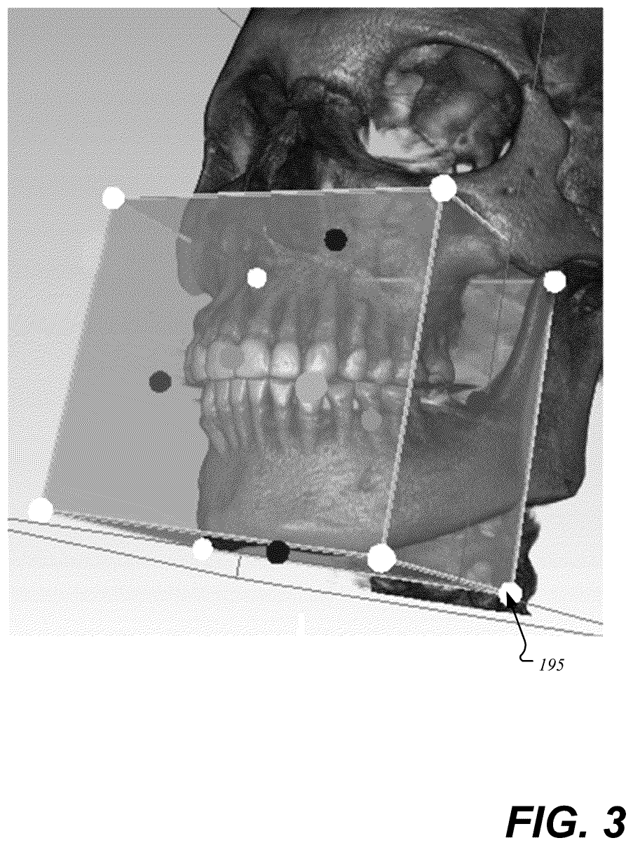

FIG. 3 is an illustration of a jaw region of interest (ROI) in a volumetric image, according to an embodiment of the present disclosure. In an embodiment, the entire dental image can be analyzed or processed at the cost of additional computational time and computational resources, such as processor power (e.g., a central processing unit (CPU), a graphics processing unit (GPU), etc.). Notably, the entire dental image need not be analyzed and a portion of the dental image can be analyzed instead. To this end, and with reference to optional step 110 of FIG. 1, a jaw ROI 195 can be determined from the dental image. The jaw ROI 195 can encompass, for example, an upper jaw, a lower jaw, and teeth attached to one or more of the jaws. The jaw ROI 195 can therefore encompass a subset of the entire dental image data to be analyzed. Analyzing the jaw ROI 195 can reduce the computational time by, for example, more than 1%, or more than 10%, or more than 20%, or more than 25%, or more than 50%, or more than 75%, or by a ratio of the jaw ROI 195 to the volumetric image, or by a ratio of the jaw ROI 195 to the 2D reconstructed image. It may be appreciated that the entire dental image data can be analyzed if desired or needed.

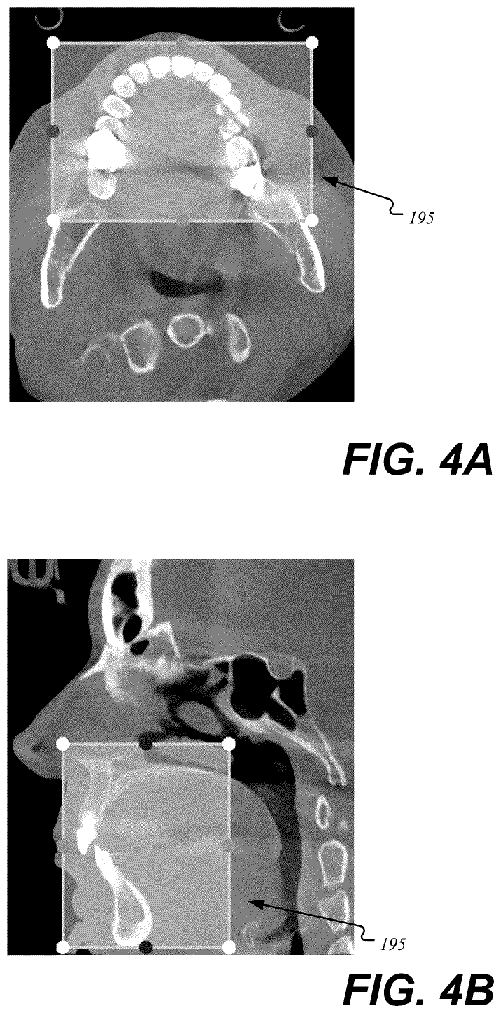

FIG. 4A is an illustration of the jaw ROI 195 in a 2D image, according to an embodiment of the present disclosure. Similarly, FIG. 4B is an illustration of the jaw ROI 195 in a 2D image, according to an embodiment of the present disclosure. In an embodiment, the 3D representation of the jaw ROI 195 in FIG. 3 can be shown as a corresponding 2D representation in FIGS. 4A and 4B. Notably, the jaw ROI 195 shown encompasses the upper jaw, lower jaw, and teeth. The jaw ROI 195 can be determined using a detection process or model. For example, the model can be 3D RetinaNet. 3D RetinaNet can be an object detection model for 3D point clouds based on the idea of single-stage object detection and use a combination of 3D feature extraction and a 2D projection of the point cloud to identify objects. 3D RetinaNet can have a multi-scale feature extraction network, an efficient region proposal network, and a detection head that uses a focal loss to identify objects. 3D RetinaNet can be used to detect objects in CT images, such as the dental image. The detection process, such as 3D RetinaNet, can first extract 3D features from the CT images. Then, regions of interest detected by the detection process and a detection head can be used to identify the objects, such as the teeth and jaws, where a detection head is a component of a neural network architecture that is responsible for predicting the class label and location of objects in an input image. A post-processing step can then be applied to refine the detections. The output can be the jaw ROI 195 coordinates, such as x_min, y_min, z_min, x_max, y_max, and z_max, as well as the visualizations shown in FIGS. 3, 4A, and 4B. The training dataset can be, for example, other CT images on the order of tens, hundreds, thousands, or more images. For example, 3000 CT images can be used to train the jaw ROI detection model over 55 hours on an NVIDIA Tesla V100 GPU.

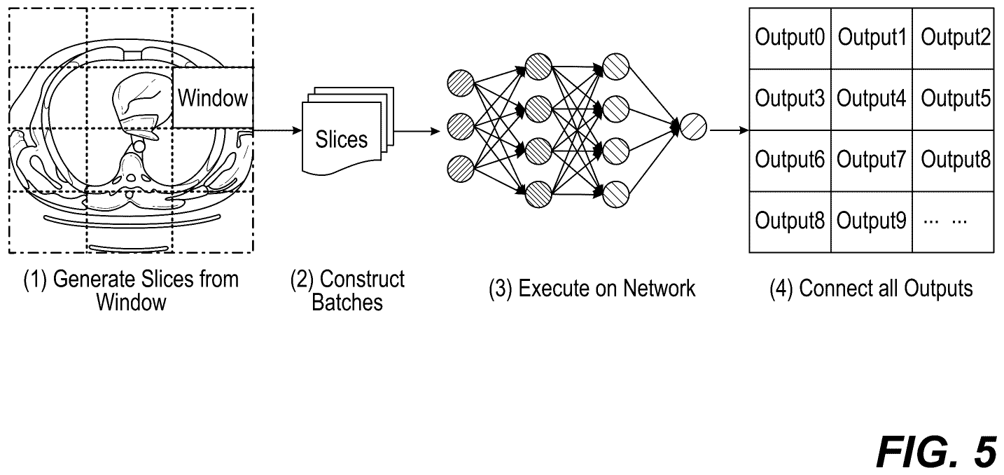

FIG. 5 is an illustration of a sliding window inference process, according to an embodiment of the present disclosure. In an embodiment, the segmentation models described herein can use a sliding window during inference for improved efficiency. The sliding window inference process can be a method used for medical image segmentation, especially for CT images. The sliding window inference process can include dividing the input image into overlapping patches or sub-regions, which are then used as the input for the segmentation model for processing. In the context of CT image segmentation, the input image can be a 3D volume including a stack or composite of 2D image slices. The segmentation model can take each slice as the input and generate a binary mask that indicates the location of different anatomical structures or lesions. The sliding window inference process can slide or translate a window of a fixed size (e.g., 64×64×64 voxels) over the input image with a predetermined stride (e.g., 32 voxels). At each location, the segmentation model can process the sub-region enclosed or encapsulated by the window and generate a corresponding binary mask. The stride can determine the degree of overlap between adjacent windows, which helps to reduce artifacts at the edges of the sub-regions. Thus, the predetermined stride can be determined by the desired degree of overlap between adjacent windows. The reduction in artifacts can be at the cost of increased processing power needed. After processing all the windows, the individual binary masks can be assembled into a final segmentation map by, for example, averaging or voting over the overlapping regions. This can help to improve the accuracy of the segmentation, especially for cases where the boundaries between different structures are ambiguous or distinct.

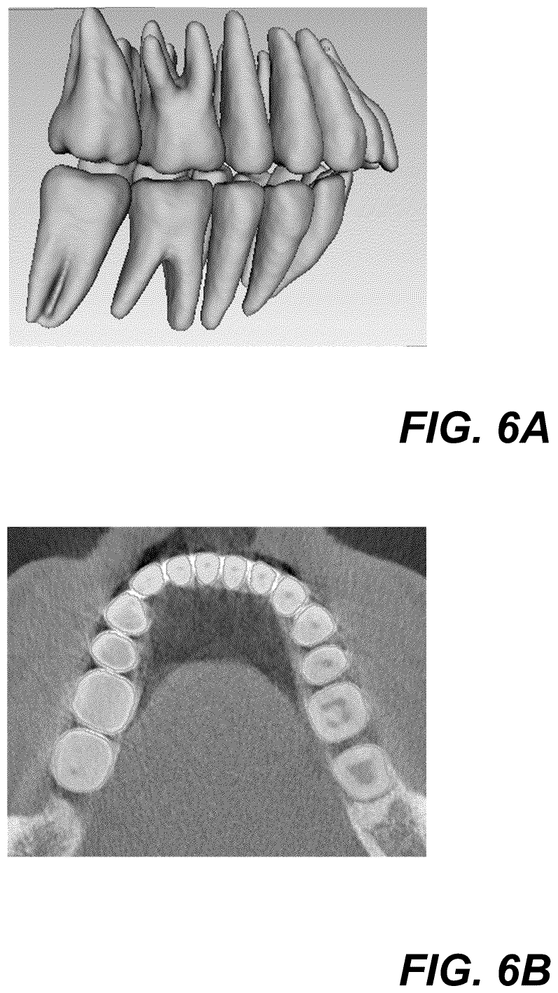

FIG. 6A is an illustration of a training image for a teeth segmentation model, according to an embodiment of the present disclosure. Similarly, FIG. 6B is an illustration of a training image for a teeth segmentation model, according to an embodiment of the present disclosure.

In an embodiment, the teeth segmentation model can use a training data set as the input to the teeth segmentation model to segment or determine locations of teeth in the input and train the teeth segmentation model. For example, the training data set can include images of rescaled and normalized jaw ROI CT images. The images can be obtained by the following process: the 3D models can be mapped onto a segmentation mask using a mesh voxelization process, and then technicians or experts can validate the data obtained. Importantly, the 3D models mapped onto the segmentation mask can include teeth location labels that the teeth segmentation model observes as ground truth data points for training because the labels have been verified by the technicians or experts. Any teeth location labels that were generated and marked as inaccurate can also be used to train the teeth segmentation model to identify and also mark as inaccurate (and therefore not label as a location of a tooth). Additional images with labels/locations verified by the technicians can serve to further increase the labeling/locating accuracy of the teeth segmentation model. The same can be true for training data used for the adhesion location model described below. The model can be, for example, SegResNet. SegResNet can be a deep neural network architecture designed for instance segmentation of medical images, including CT scans. The model can be based on the ResNet architecture, which uses residual connections to overcome the problem of vanishing gradients in deep networks. SegResNet can combine the ResNet architecture with a fully convolutional network (FCN) approach to achieve instance segmentation, where each object in the image is assigned a unique label.

In an embodiment, the model can use an encoder-decoder architecture, where the encoder part is based on the ResNet architecture, and the decoder part is an FCN. The encoder can take the input image and process it through a series of convolutional layers, downsampling the image at each step to extract increasingly abstract features. The decoder can then take the extracted features and upsample them to the original resolution, using transposed convolutional layers. In addition to the ResNet-based encoder-decoder architecture, SegResNet can also incorporate skip connections between the encoder and decoder, which allow the model to fuse information from multiple levels of the feature hierarchy. This can help the model to better localize and segment objects, especially in cases where the objects are partially occluded or have complex shapes. SegResNet can be trained using a combination of supervised and unsupervised methods, where the supervised component involves minimizing the cross-entropy loss between the predicted and ground-truth segmentation masks. The unsupervised component involves minimizing the distance between the encoded and decoded features, which helps to enforce consistency and stability in the learned representations. Overall, SegResNet is a state-of-the-art model for instance segmentation of medical images, including CT scans. SegResNet has been shown to achieve high accuracy and robustness and has the potential to facilitate a wide range of applications in medical imaging and diagnosis.

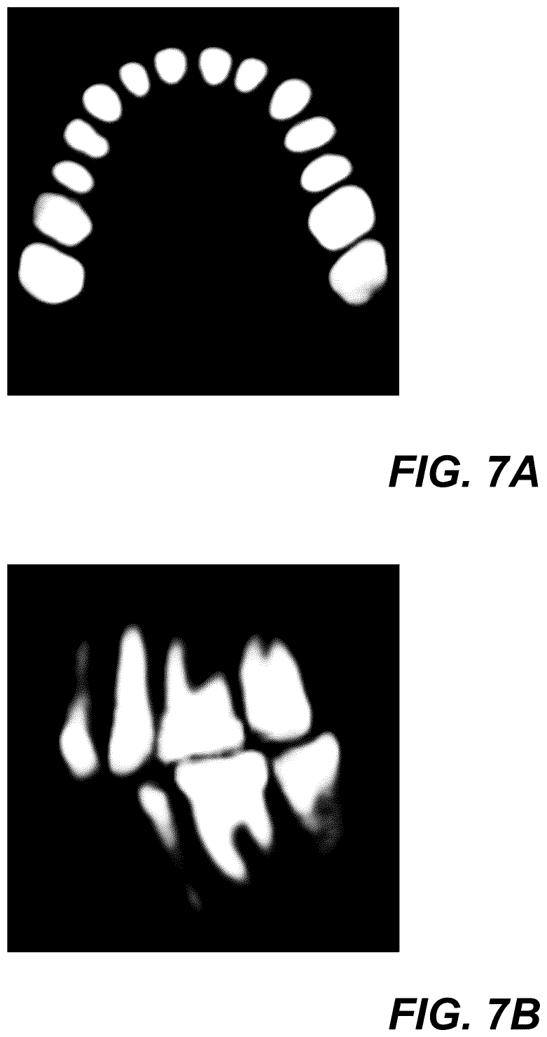

In an embodiment, and with reference to step 115 of FIG. 1, the obtained dental image and/or the jaw ROI 195 can be used as an input to the teeth segmentation model to segment or determine locations of teeth in the input. For example, the jaw ROI 195 determined from the obtained dental image can be used as the input and the teeth segmentation model can segment the patient's teeth in the jaw ROI 195. That is, the teeth segmentation model can identify individual teeth and the locations of the individual teeth in the jaw ROI 195. The segmentation or locations can be output as a probability map described below with reference to FIGS. 7A and 7B.

FIG. 7A is an illustration of the probability map which can be generated via the teeth segmentation model using the obtained dental image or the jaw ROI 195 as an input, according to an embodiment of the present disclosure. Similarly, FIG. 7B is an illustration of a probability map 790 which can be generated via the teeth segmentation model using the obtained dental image or the jaw ROI 195 as an input, according to an embodiment of the present disclosure. In an embodiment, the output of the model can be the probability maps describing the location of the individual teeth in the jaw ROI 195 when the jaw ROI 195 is used as the input for the teeth segmentation model. That is, the (3D) jaw ROI 195 can include measurements of radiation attenuation for radiation detected by a detector along a line of response (LOR) through the patient. The teeth segmentation model can use these values to generate the likelihood that, based on the attenuation value at a predetermined voxel in the jaw ROI 195, the material at the predetermined voxel corresponds to a tooth, a jaw bone, or cheek tissue. A similar process can be applied to 2D images or slices and predetermined pixels in the 2D slice of the jaw ROI 195.

For example, the output of SegResNet can be a pixel-wise segmentation map of the dental image, wherein each pixel in the input image is assigned a label that corresponds to the class of object or region it represents. The number of output channels in the SegResNet can depend on the number of classes in the segmentation task. For example, in a binary segmentation task (where there are only two classes: foreground and background), the output map can have only one channel, wherein each pixel can be assigned a value of either 0 or 1. In a multi-class segmentation task, there can be multiple output channels, wherein each channel corresponds to a different class label and each pixel is assigned a value between 0 and 1 representing the probability that it belongs to that class. The final output map can be obtained by selecting the class label with the highest probability for each pixel. FIGS. 7A and 7B show output probability maps of the 2D slices extracted from the obtained dental image. Here, the white pixels indicate the high probability that the material corresponds to material of a tooth.

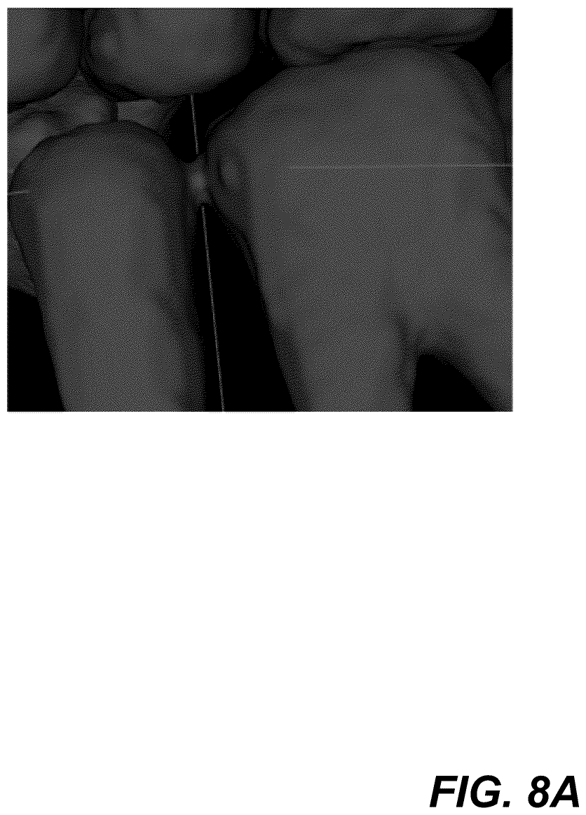

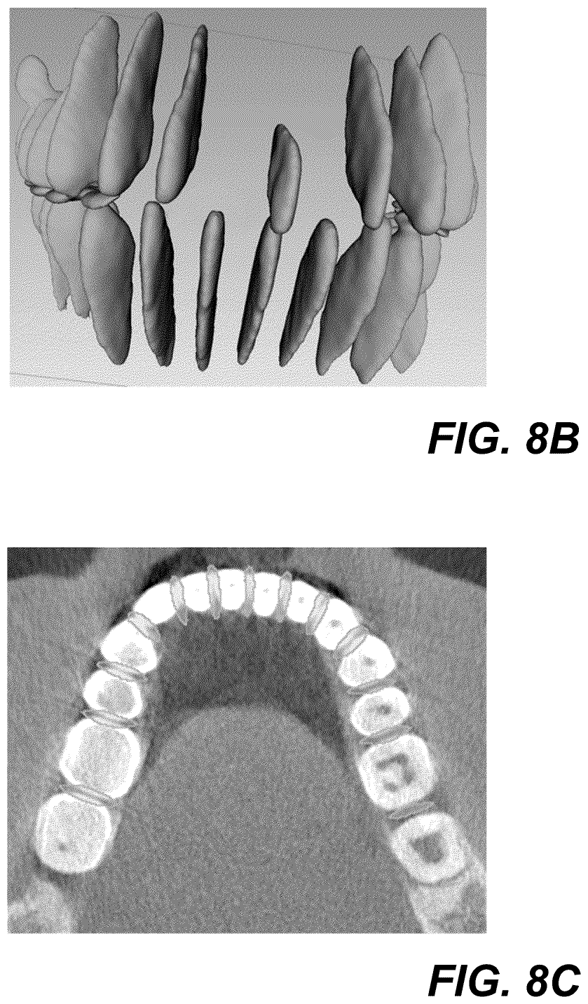

One difficult aspect of teeth segmentation can be accurate border segmentation. Due to anomalies on CT images or small distances between teeth, a segmentation model may produce an incorrect pixel classification on a border of a tooth. When generating the teeth segmentation outputs, noise can be removed from the segmentation using a classifier, and in the resulting segmentation, teeth may be stuck together due to their close proximity or the presence of anomalies in the image. FIG. 8A is an illustration of the segmentation issue in a 3D rendering, according to an embodiment of the present disclosure.

In an embodiment, to address this issue, and with reference to step 120 of FIG. 1, the method 100 in the present disclosure can use the second model to identify and determine the adhesion locations in the jaw ROI 195 using the jaw ROI 195 as an input. That is to say, locations where adjacent teeth appear “glued” or connected. The second model can herein additionally be referred to as the adhesion location, adhesion location segmentation, abutment location, or collision location model. The identified adhesion locations can be used, as described below, in combination with the probability map of the teeth segmentation model to decouple any teeth showing adhesion between one another. Additionally or alternatively, the entire obtained dental image can be used as the input.

In an embodiment, the adhesion location model can also use a synthetically generated dataset with masked possible locations of abutting or collided or adhered or stuck teeth as a training dataset. As described previously and further below, training data can be generated and then reviewed by experts or technicians. The technician can review the training data to ensure that labels identifying the adhesion locations are accurate, which will serve as the ground truth data for the model. This allows the model to further improve labeling accuracy for future input data. Then, a segmentation model, such as SegResNet, can be fit using the synthetically generated dataset. Then, at an inference phase, using the sliding window inference process, the stuck or adhered teeth locations can be identified using the output of the segmentation model, i.e., a binary segmentation mask of the adhesion locations.

To this end, FIG. 8B is an illustration of a training image for identifying adhesion locations using the adhesion location model, according to an embodiment of the present disclosure. Similarly, FIG. 8C is an illustration of a training image for identifying adhesion locations using the adhesion location model, according to an embodiment of the present disclosure. The training images can be rescaled and normalized jaw ROI CT images. Notably, training data for teeth can be balanced by gender, age, metal presence, number of teeth, and bone and dental density. The data can be divided into training/validation/test preserving data distributions.

In an embodiment, a mask for each tooth can be generated and subsequently enlarged in size by a predetermined amount. Upon the mask enlargements of a first tooth in the upper jaw and a second tooth in a lower jaw arranged opposite one another, the two enlarged masks can, at one or more locations, begin to contact and intersect one another at the adhesion or stuck locations. This process can be iterated for all of the teeth. Additionally or alternatively, upon mask enlargements of a first tooth and a second tooth in the same jaw and adjacent to one another, the two enlarged masks can, at one or more locations, begin to contact and intersect one another at the adhesion or stuck locations.

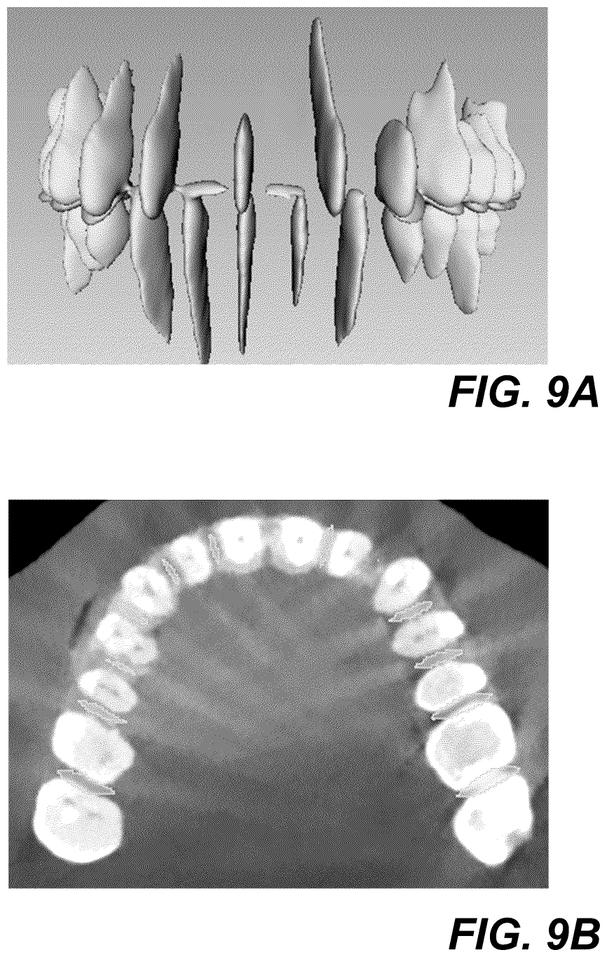

FIG. 9A is an illustration of a 3D binary segmentation mask of the adhesion locations generated via the adhesion location model using the jaw ROI 195 as an input, according to an embodiment of the present disclosure. FIG. 9B is an illustration of a 2D mask slice of the 3D binary segmentation mask of the adhesion locations generated via the adhesion location model using the jaw ROI 195 as an input, according to an embodiment of the present disclosure. The output of the adhesion location model, such as SegResNet, for the adhesion area can be, for example, a binary segmentation mask as previously described above with reference to step 120 of the method 100. As shown, the 3D binary segmentation mask and the 2D mask slice highlight areas between the teeth, which can include possible adhesion or stuck locations.

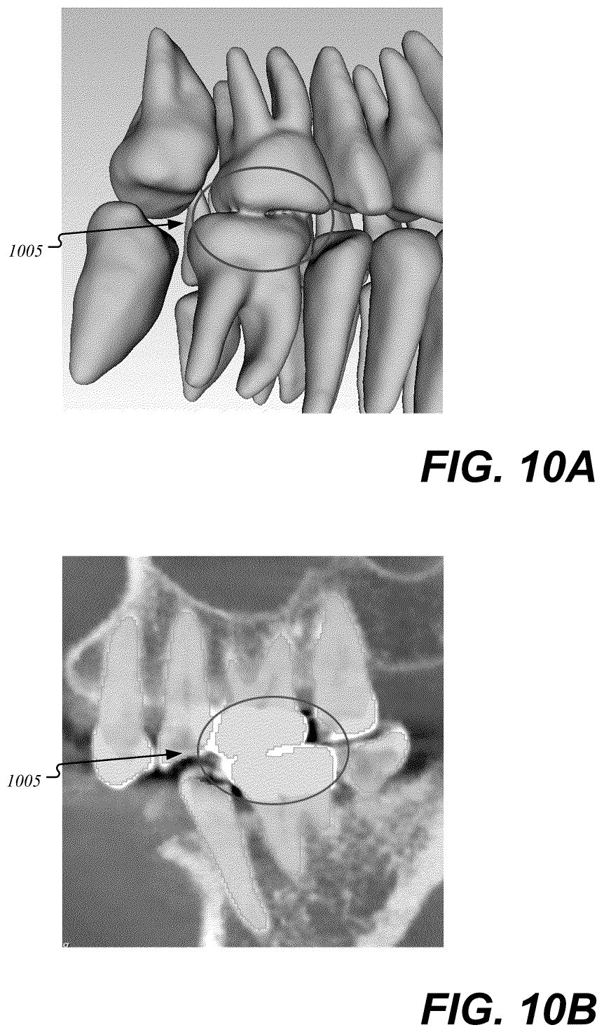

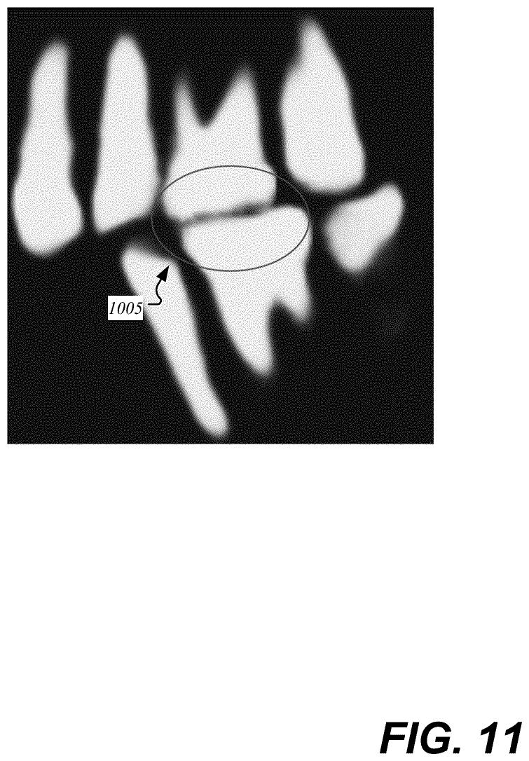

FIG. 10A is an illustration of a 3D mask of teeth segmentation showing an adhesion location, according to an embodiment of the present disclosure. FIG. 10B is an illustration of a 2D slice of the 3D mask of teeth segmentation showing an adhesion location, according to an embodiment of the present disclosure. In particular, FIGS. 10A and 10B are results of segmenting the teeth or determining locations of the individual teeth using the segmentation model (the first model), but still show lower teeth and upper teeth adhesion issues, such as at adhesion location 1005. FIG. 11 is an illustration of a probability map used as an input for a teeth separation process, according to an embodiment of the present disclosure. FIGS. 10A and 10B are the output 3D and 2D masks, and FIG. 11 is the output probability map from the teeth segmentation model with artifacts that cause the appearance of adhesion locations between teeth, such as the adhesion location 1005. In an embodiment, the input to the teeth separation process can be visualized as a 3D mask of teeth segmentation (FIG. 10A) and a 2D slice of the teeth segmentation (FIG. 10B), but in actuality, the probability map (FIG. 11) of the teeth is the true input to the teeth separation process. FIG. 11 is the probability map corresponding to the results of segmenting the teeth, which also shows the adhesion location 1005.

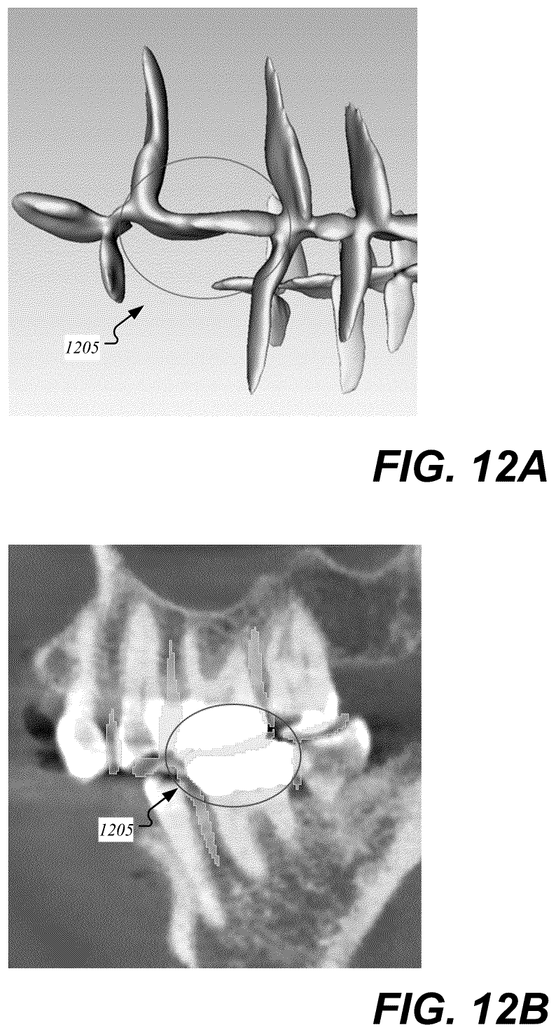

FIG. 12A is an illustration of an input for a teeth separation process, according to an embodiment of the present disclosure. FIG. 12B is an illustration of an input for a teeth separation process, according to an embodiment of the present disclosure. FIG. 12B illustrates, specifically, a binary mask output of the adhesion location model overlaid on the CT image. In particular, FIGS. 12A and 12B are results of determining locations of the adhesions between teeth. The binary mask output of the adhesion location model was generated by obtaining a CT image, generating a mask for each tooth, enlarging the mask in size by a predetermined amount until the masks for the teeth began to intersect, and identifying the intersection areas or volumes for inclusion in the binary mask. While all teeth adhesion locations are determined during the inference phase of the adhesion location segmentation model, some adhesion locations can be categorized or grouped according to a particular occurrence, such as between different sets of teeth. For example, FIGS. 12A and 12B highlight one such group that can be described as an upper-lower teeth adhesion location 1205, which is an adhesion location occurring between the upper teeth and the lower teeth. As previously noted, noise can be removed from the segmentation using a classifier, and in the resulting segmentation, teeth may be stuck together due to their close proximity or the presence of anomalies in the image.

To remove adhesions, masks of the possible presence of adhesions are built and, using the prediction probabilities of the previous model (the teeth segmentation), decouples the teeth. In an embodiment, the teeth separation process performs a binary search using a threshold from [0.5, 1], where the threshold is a standard threshold for a classification task and is applied to the probability map of the teeth segmentation inside the adhesion location for each connected/adhered component of the adhesion location until all teeth are separated.

In an embodiment, the inputs of the teeth separation process (step 125) can be: (i) the probability map output of the teeth segmentation model (step 115, the first model), for example FIG. 11, and (ii) the binary mask output of the adhesion location model (step 120, the second model), for example FIG. 12B. Notably, the mask in FIG. 12B is shown overlaid on a CT image.

In order to produce more accurate results, the binary adhesion mask locations can be divided into a set of connected components. Then, a process described herein can be applied to each of the connected components. A component of the connected components can be termed, for example, ADHcomp.

In an embodiment, the connected component, or one of the identified masses (or volumes), in a binary image (the binary adhesion mask) can be defined by a set of adjacent pixels (or voxels). Determining which pixels are adjacent depends on how pixel connectivity is defined. For a two-dimensional image, there are two standard connectivities. A first connectivity type is a 4-connectivity, where pixels are connected if their edges touch. Two adjoining pixels are part of the same object if they are both on and are connected along the horizontal or vertical direction. A second connectivity type is a 8-connectivity, where pixels are connected if their edges or corners touch. Two adjoining pixels are part of the same object if they are both on and are connected along the horizontal, vertical, or diagonal direction. It may be appreciated that nonstandard connectivities can be defined, or connectivities for higher dimensional images, e.g., 3D images.

In an embodiment, the connected component of the binary mask of the adhesion locations (ADHcomp) can be applied to the probability map output of the teeth segmentation model to generate a probability map of predetermined possible locations of teeth adhesion. That is, the binary mask components can further define portions of the probability map where there is a high likelihood of teeth adhesion. The application of the binary mask of the adhesion locations to the probability map output of the teeth segmentation model can include performing, for example, a Hadamard product (an element-wise product) of the binary mask of the adhesion locations and the probability map output of the teeth segmentation model. That is, an element (i,j) of the binary mask can be multiplied by an element (i,j) of the probability map to yield an element (i,j) of a new mask and repeated for integers from {[0, m), [0, n)], where m and n define dimensions or a size of the probability map and the binary mask. That is, each element of the binary mask can be multiplied by a corresponding element of the probability map to generate a new image having the same number of elements and therefore the same size as the input binary mask and probability map. The product of the two elements can be located at the same corresponding location in the new mask.

Then, a threshold can be applied to decouple teeth from one another if they are stuck or adhered. The threshold value can be a value from 0 to 1. For example, a threshold value of 1 can be selected (i.e., leave only pixels/voxels with a probability greater than or equal to 1) and applied to the probability map of the predetermined possible locations of teeth adhesion. With this threshold value, for each pixel (or voxel) of the probability map, the probability value of that pixel is compared to the threshold value. If this probability value is greater, then a value of 1 is assigned to the pixel (or voxel). If this probability is value is lower, then a value of 0 is assigned to the pixel. That is, the threshold value can be selected as a cut-off where pixels or voxels having a probability greater than the threshold value are retained in the resulting image, while pixels or voxels having a probability lower than the threshold value are excluded. Therefore, after thresholding, a new binary mask is obtained that further describes the adhesion locations and size of the adhesion locations for a given threshold value. However, this can result in removal of large portions of teeth depending on the threshold value selected. Thus, a binary search process can be used.

The binary search can proceed as followed. First, for example, a number A of connected components are counted in the probability map for a first threshold value. For example, in general terms, the teeth separation process can identify 3 teeth in an upper jaw adjacent to one another, 3 teeth in a lower jaw adjacent to one another, and an additional tooth in the upper jaw plus an additional tooth in the lower jaw, but the additional tooth in the upper jaw and the additional tooth in the lower jaw share an adhesion location and thus appear connected prior to decoupling. As such, the number A for a successful decoupling is determined to be 8 (4 teeth in the upper jaw and 4 teeth in the lower jaw). In contrast, the number A for an unsuccessful decouple is determined to be 7 connected components (6 masses for the 3 teeth in the upper jaw and the 3 teeth in the lower jaw, plus the 1 mass for the 2 teeth that remain connected).

Then, for example, a second threshold value can be selected and the binary mask of the adhesion locations can be applied to the probability map to decouple the teeth. The second threshold value can be lower than the first threshold value. The number B of connected components are determined again. For example, the 1 mass for the 2 connected teeth can be successfully decoupled, thus resulting in 2 masses for a total number B of 8. Notably, since the second threshold value is lower than the first threshold value, the amount of actual teeth material removed during the decoupling can be less. Therefore, the second threshold value provides better decoupling (less teeth material removed) while retaining the minimum accuracy in order to successfully identify an adhesion location and remove it/decouple teeth.

Additionally or alternatively, for example, a third threshold value can be selected and the binary mask of the adhesion locations can be applied to the probability map to decouple the teeth. The third threshold value can be less than the second threshold value. The number B of connected components are determined again. For example, the 1 mass for the 2 connected teeth can be unsuccessfully decoupled, thus resulting in 1 mass for a total number B of 7. Notably, since the third threshold value is lower than the second threshold value, the amount of actual teeth material removed during the decoupling can be even less, but in this case, too little. This leaves the two teeth connected because of the adhesion location that was not identified and removed. Therefore, the third threshold value provides worse decoupling. As such, the second threshold value provides the best decoupling between the three threshold values used. This process can be iterated for additional threshold values based on the desired level of optimization of the threshold value.

In an embodiment, the adhesion locations can be predicted using a trained adhesion location segmentation model. In order to train the adhesion location segmentation model, CT images with possible adhesion locations included therein can be labeled synthetically to identify the possible adhesion locations to generate labeled images or training images. In order to generate labeled images with adhesion locations (masks), labeled CT images with teeth locations (masks) are used. For each of the teeth in the binary mask, the mask of the teeth are enlarged or dilated by a predetermined amount. More than one size adjustment can be performed on the masks with varying size adjustments, such as enlargements of 1%, 5%, 10%, 25%, or more. Further, the enlargements can be along a single dimension or two dimensions instead of being a uniform enlargement. The locations where these enlarged teeth (their masks) have intersected are the possible locations of teeth adhesion. The segmentation models can be trained on such labeled images and data to detect these locations more accurately during the inference phase with a clinical, non-training image. A similar process can be applied to the clinical, non-training image, but instead, the trained adhesion location segmentation model can generate the labels/adhesion locations that are typically provided during the training phase. In this manner, a more extensively trained adhesion location segmentation model can more accurately identify, locate, and label any adhesion locations in the clinical image.

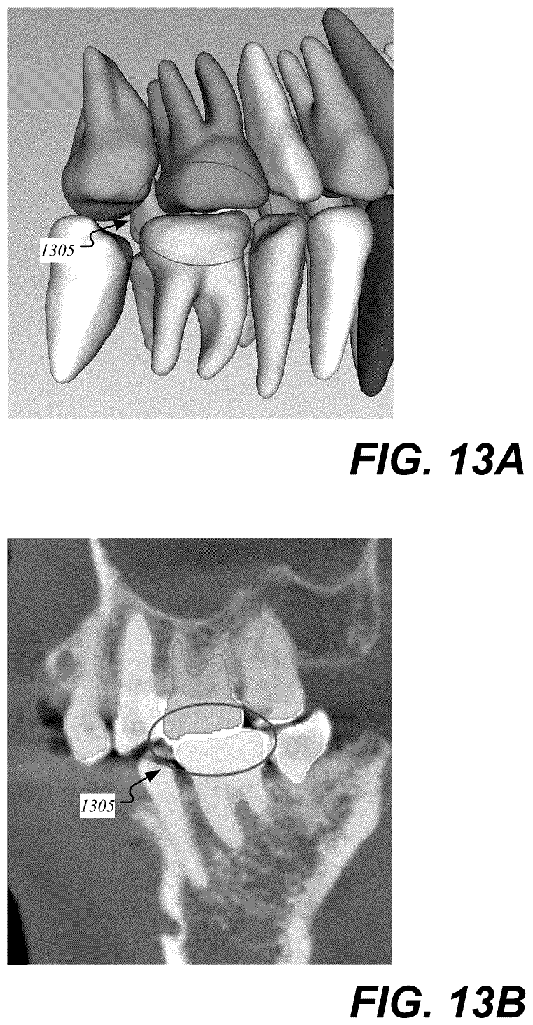

FIG. 13A is an illustration of an output of a teeth separation process, according to an embodiment of the present disclosure. FIG. 13B is an illustration of an output for a teeth separation process, according to an embodiment of the present disclosure. FIG. 13A is a 3D model rendered based on a 3D mask generated by the teeth separation process that describes the decoupled teeth. In an embodiment, and with reference to step 125 of FIG. 1, the 3D model of FIG. 13A and the 2D slice of FIG. 13B show that the adhesions between the upper teeth and the lower teeth have been identified and decoupled. In other words, the teeth separation process can generate a 3D model of decoupled teeth (a decoupled teeth 3D model) based on the determined 3D mask, as well as a 2D image of decoupled teeth. These are shown in FIGS. 13A and 13B as decoupled upper-lower location 1305. For example, the teeth separation process can use the probability map of the teeth segmentation model from FIG. 11 and the binary segmentation mask based on the adhesion location model from FIG. 12B to determine the adhesion locations between the upper teeth and the lower teeth and decouple the teeth at the determined adhesion locations to generate the decoupled upper-lower location 1305.

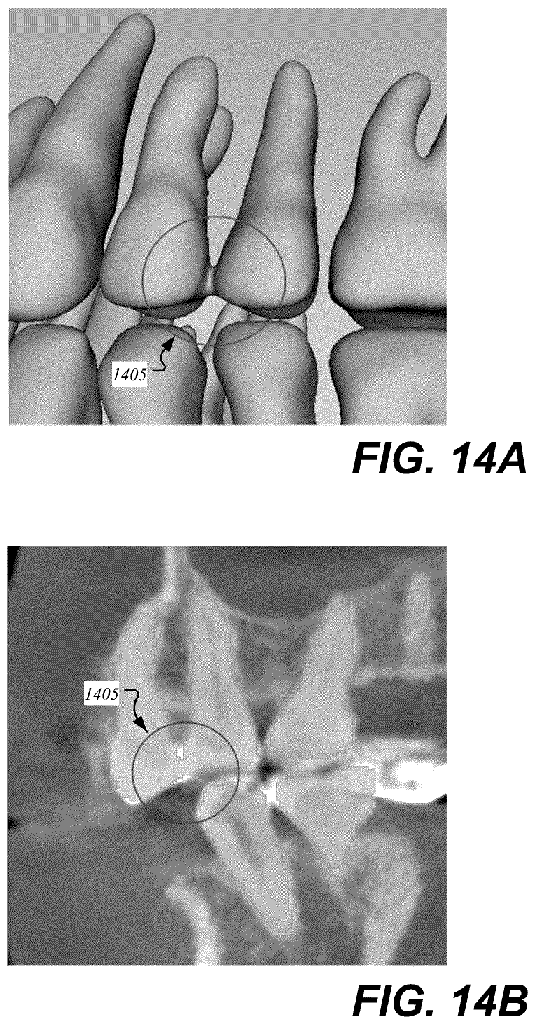

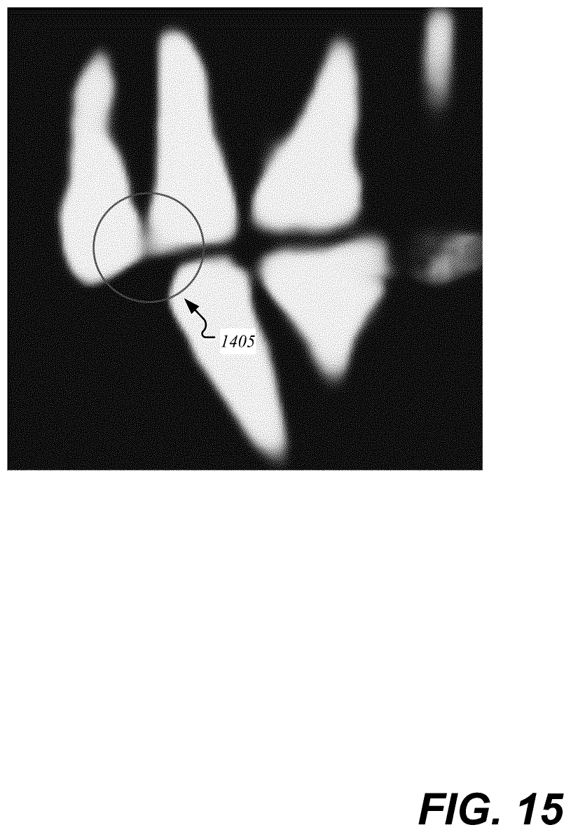

FIG. 14A is an illustration of a 3D mask of teeth segmentation showing an adhesion location, according to an embodiment of the present disclosure. FIG. 14B is an illustration of a 2D slice of the 3D mask of teeth segmentation showing an adhesion location, according to an embodiment of the present disclosure. In particular, FIGS. 14A and 14B are results of segmenting the teeth or determining locations of the individual teeth, but still show adhesion issues between neighboring teeth in the lower teeth and adhesion issues between neighboring teeth in the upper teeth, such as at adhesion location 1405 between two teeth in the upper teeth. FIG. 15 is an illustration of a probability map used as an input for a teeth separation process, according to an embodiment of the present disclosure. FIGS. 14A and 14B are the output 3D and 2D masks, and FIG. 15 is the output probability map from the teeth segmentation model with artifacts that cause the appearance of adhesion locations between teeth, such as the adhesion location 1405. In an embodiment, the input can be visualized as a 3D mask of teeth segmentation (FIG. 14A) and a 2D slice of the teeth segmentation (FIG. 14B), but in actuality, the probability map (FIG. 15) of the teeth is the true input. FIG. 15 is the probability map corresponding to the results of segmenting the teeth, which also shows the adhesion location 1405.

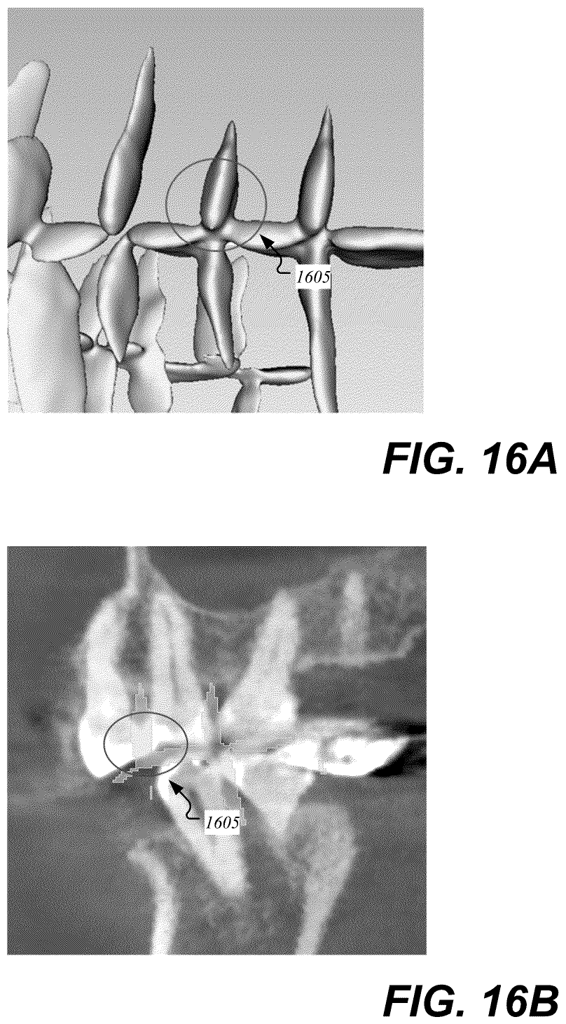

FIG. 16A is an illustration of an input for a teeth separation process, according to an embodiment of the present disclosure. FIG. 16B is an illustration of an input for a teeth separation process, according to an embodiment of the present disclosure. In particular, FIGS. 16A and 16B are results of determining locations of the adhesions between teeth. While all teeth adhesion locations are determined during the inference phase of the adhesion location segmentation model, some adhesion locations can be categorized or grouped according to a particular occurrence, such as between different sets of teeth. For example, FIGS. 16A and 16B highlight one such group that can be described as a neighboring teeth adhesion location 1605, which is an adhesion location occurring between two adjacent teeth in the same jaw or row of teeth. As previously noted, noise can be removed from the segmentation using a classifier, and in the resulting segmentation, teeth may be stuck together due to their close proximity or the presence of anomalies in the image.

As previously described, to remove adhesions, masks of the possible presence of adhesions are built and, using the prediction probabilities of the previous model (the teeth segmentation), decouples the teeth. In an embodiment, the teeth separation process performs a binary search using a threshold from [0.5, 1], where the threshold is a standard threshold for a classification task and is applied to the probability map of the teeth segmentation inside the adhesion location for each connected/adhered component of the adhesion location until all teeth are separated. The same training phase process and inference phase process can be used as described previously. Here, in an embodiment, since adhesion locations between neighboring teeth are of interest, the masks of the teeth can be enlarged or adjusted along the width dimension of the teeth.

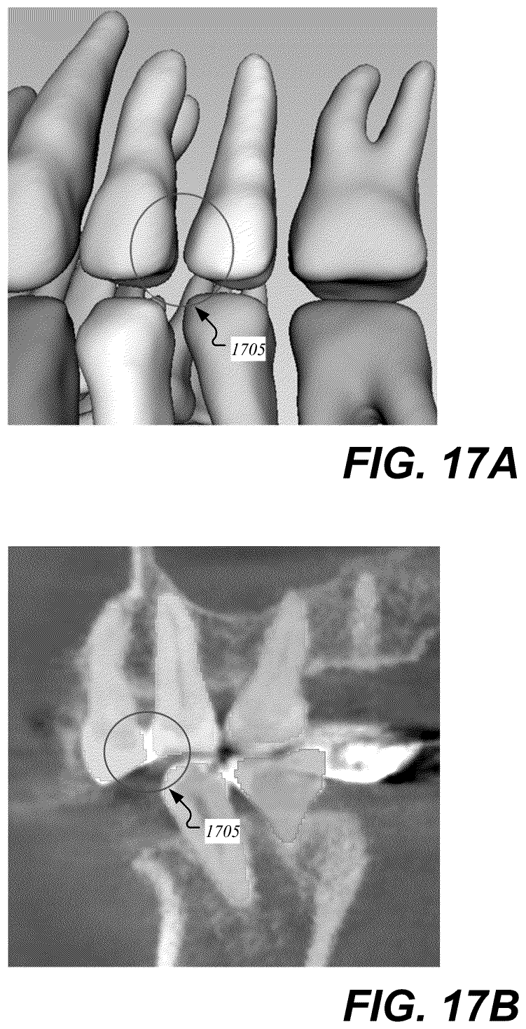

FIG. 17A is an illustration of an output of a teeth separation process, according to an embodiment of the present disclosure. FIG. 17B is an illustration of an output for a teeth separation process, according to an embodiment of the present disclosure. FIG. 17A is a 3D model rendered based on a 3D mask generated by the teeth separation process that describes the decoupled teeth. In an embodiment, and with reference to step 125 of FIG. 1, the 3D model of FIG. 17A and the 2D slice of FIG. 17B show that the adhesions between the neighboring teeth along the same row of teeth have been identified and decoupled. In other words, the teeth separation process can generate a 3D model of decoupled teeth based on the determined 3D mask, as well as a 2D image of decoupled teeth. These are shown in FIGS. 17A and 17B as decoupled neighboring location 1705. For example, the teeth separation process can use the probability map of the teeth segmentation model from FIG. 15 and the binary segmentation mask based on the adhesion location model from FIG. 16B to determine the adhesion locations between the neighboring teeth and decouple the teeth at the determined adhesion locations to generate the decoupled neighboring location 1705.

In an embodiment, the outputs of the upper and lower teeth adhesion locations, the neighboring teeth adhesion locations, and the teeth segmentation can be used to improve the accuracy of determining locations of individual teeth from dental images.

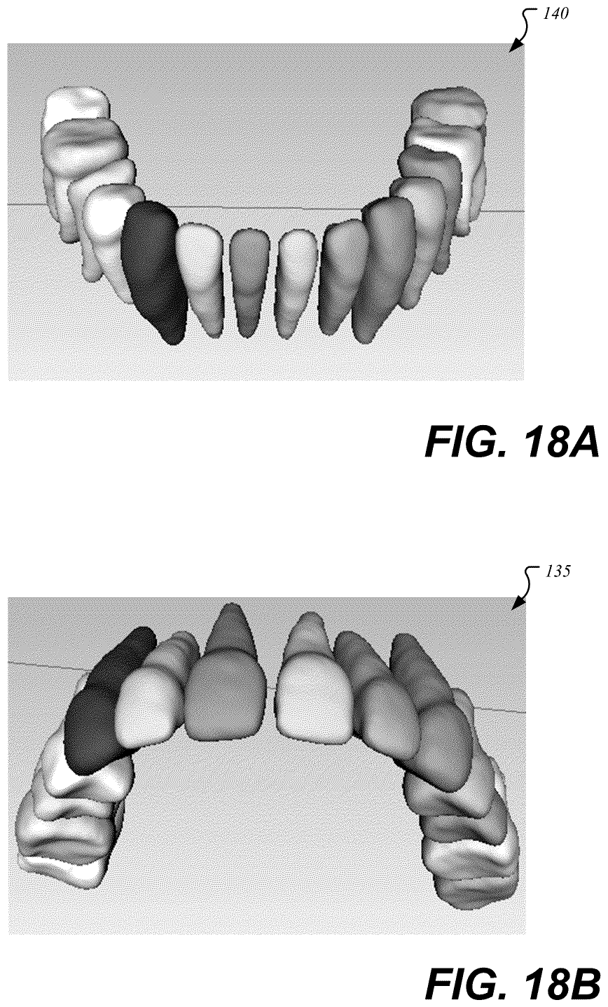

Finally, FIG. 18A is an illustration of an output for a teeth splitting process, according to an embodiment of the present disclosure. Similarly, FIG. 18B is an illustration of an output for a teeth splitting process, according to an embodiment of the present disclosure. In an embodiment, and with reference to step 130 of FIG. 1, once the individual teeth locations have been determined, the teeth can be split via the teeth splitting process into the upper jaw and the lower jaw based on, for example, a proximity of the teeth to a respective upper jaw bone or lower jaw bone. FIG. 18A shows the teeth determined to be disposed in the lower jaw as the lower jaw teeth binary mask 140, and FIG. 18B shows the teeth determined to be disposed in the upper jaw as the upper jaw teeth binary mask 135. A Marching cube process can be applied to generate the 3D models.

In an embodiment, orthodontic treatment devices, also known as dental aligners, can be developed for one or more stages of teeth movement, which can be planned movement or adjustment of a patient's teeth towards a predetermined target dental arch. The dental aligners can be for an upper dental arch and a lower dental arch. Phases or checkpoints of teeth movement can be split into stages, each stage defining and providing an arrangement of the teeth for an orthodontic treatment device fabricated therefor. An accurate determination of the teeth locations at the starting stage via the obtained dental image (CT scan) can provide the best starting point for the overall treatment plan, and importantly, provide the most comfortable and best-fitting dental aligner for the patient. That is, an updated mask or 3D model of the teeth imaged during the CT scan with adhesion locations eliminated from the mask can be generated (the decoupled teeth 3D model), and from this updated mask or 3D model a more accurate dental aligner can be fabricated. The adhesion locations can significantly decrease or entirely inhibit the fit of the dental aligner if not identified and removed during the planning or prediction phases of the treatment plan. Therefore, the method described herein can provide an important advantage in more accurately analyzing dental images to eliminate the adhesion locations present. Similarly, obtaining additional CT scans at subsequent teeth movement stages can be performed in order to update or correct the predicted teeth movements and locations at said stages, and additional image processing at said stages to eliminate any adhesion locations in the dental images can further help improve the accuracy of the dental aligners fabricated for each stage.

In an embodiment, the decoupled teeth 3D model can be used to generate a mold or a series of molds, which can then, in turn, be used to form the aligners. The series of molds can include one mold per stage of teeth movement. The progression of the decoupled teeth 3D model from original, starting teeth positions to final, desired teeth positions can be generated automatically or via a technician, or a combination of both. Notably, a decoupled teeth 3D model can be generated per stage. Once the stages are finalized, the corresponding molds for each stage can be fabricated. For example, an additive manufacturing process, such as stereolithography, can be used to fabricate the molds. The molds can be used to form the aligners via, for example, a thermoforming process. A polymer, such as a thermoplastic, can be heated above a predetermined temperature, such as a glass transition temperature where the thermoplastic is in an amorphous state. Once heated, the polymer can be formed over the mold in order to adopt the features of the mold (i.e., the teeth topology). This can be achieved via, for example, a vacuum thermoforming process. The polymer can be cooled, and the negative shape of the mold can be locked in for further downstream processing, such as smoothing and polishing.

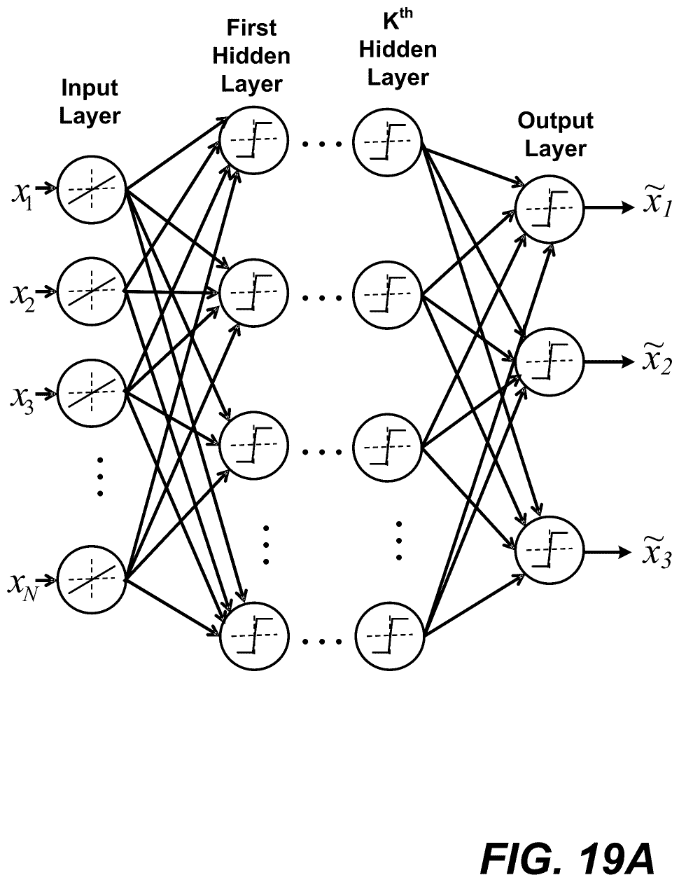

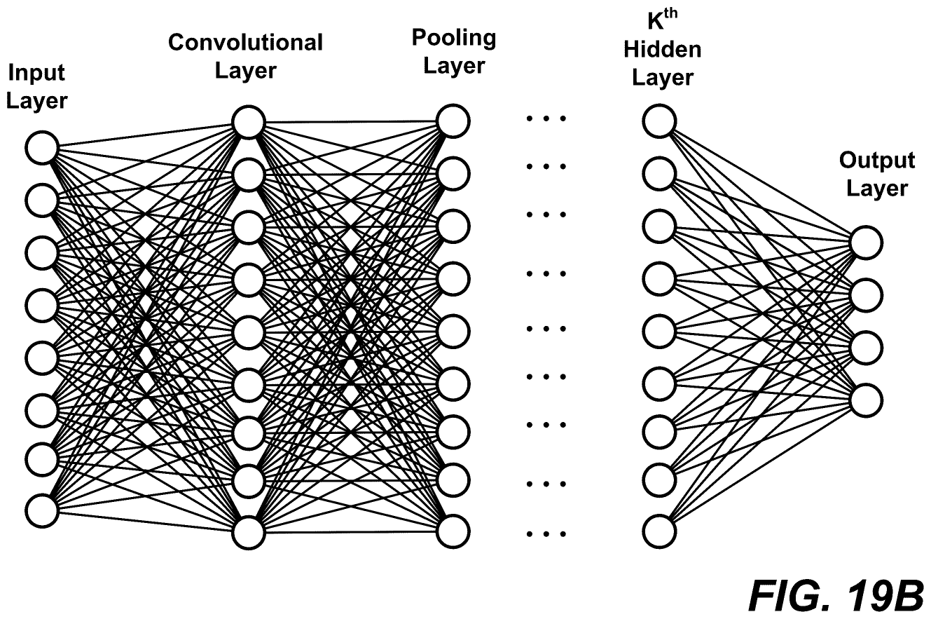

FIGS. 19A and 19B show various examples of a deep learning (DL) network.

FIG. 19A shows an example of a general artificial neural network (ANN) having N inputs, K hidden layers, and three outputs. Each layer is made up of nodes (also called neurons), and each node performs a weighted sum of the inputs and compares the result of the weighted sum to a threshold to generate an output. ANNs make up a class of functions for which the members of the class are obtained by varying thresholds, connection weights, or specifics of the architecture such as the number of nodes and/or their connectivity. The nodes in an ANN can be referred to as neurons (or as neuronal nodes), and the neurons can have inter-connections between the different layers of the ANN system. The simplest ANN has three layers, and is called an autoencoder. The DL network generally has more than three layers of neurons, and has as many outputs neurons \tilde{x}_{N} as input neurons, wherein N is the number of pixels in the reconstructed image (sinogram). The synapses (i.e., the connections between neurons) store values called “weights” (also interchangeably referred to as “coefficients” or “weighting coefficients”) that manipulate the data in the calculations. The outputs of the ANN depend on three types of parameters: (i) the interconnection pattern between the different layers of neurons, (ii) the learning process for updating the weights of the interconnections, and (iii) the activation function that converts a neuron's weighted input to its output activation.

Mathematically, a neuron's network function m(x) is defined as a composition of other functions nᵢ(x), which can further be defined as a composition of other functions. This can be conveniently represented as a network structure, with arrows depicting the dependencies between variables, as shown in the figures. For example, the ANN can use a nonlinear weighted sum, wherein m(x)=K(Σᵢwᵢnᵢ(x)), where K (commonly referred to as the activation function) is some predefined function, such as the hyperbolic tangent.

In FIG. 19A (and similarly in FIG. 19B), the neurons (i.e., nodes) are depicted by circles around a threshold function. For the non-limiting example shown in FIG. 19A, the inputs are depicted as circles around a linear function, and the arrows indicate directed connections between neurons. In certain implementations, the DL network is a feedforward network as exemplified in FIGS. 19A and 19B (e.g., it can be represented as a directed acyclic graph).

The DL network 135 operates to achieve a specific task, such as denoising a CT image, by searching within the class of functions F to learn, using a set of observations, to find m*∈F which solves the specific task in some optimal sense. For example, in certain implementations, this can be achieved by defining a cost function C:F→m such that, for the optimal solution m*, C(m*)≤C(m)∀m∈F (i.e., no solution has a cost less than the cost of the optimal solution). The cost function C is a measure of how far away a particular solution is from an optimal solution to the problem to be solved (e.g., the error). Learning algorithms iteratively search through the solution space to find a function that has the smallest possible cost. In certain implementations, the cost is minimized over a sample of the data (i.e., the training data).

FIG. 19B shows a non-limiting example in which the DL network is a convolutional neural network (CNN). CNNs are type of ANN that has beneficial properties for image processing, and, therefore, have specially relevancy for the applications of image denoising and sinogram restoration. CNNs use feed-forward ANNs in which the connectivity pattern between neurons can represent convolutions in image processing. For example, CNNs can be used for image-processing optimization by using multiple layers of small neuron collections which process portions of the input image, called receptive fields. The outputs of these collections can then tiled so that they overlap, to obtain a better representation of the original image. This processing pattern can be repeated over multiple layers having alternating convolution and pooling layers.

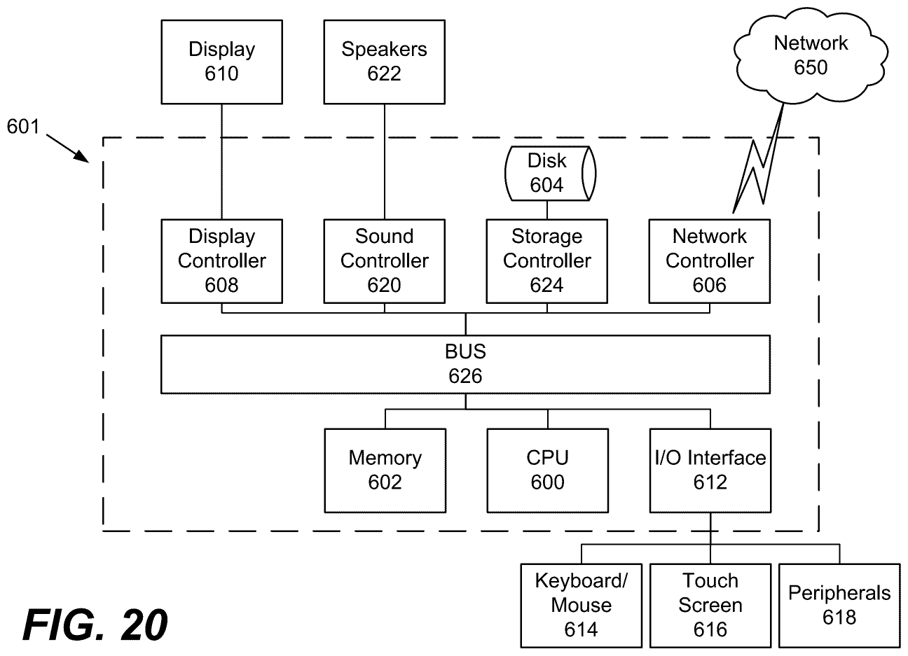

Next, a hardware description of a device 601 for performing the aforementioned methods and processes according to exemplary embodiments is described with reference to FIG. 20. In FIG. 20, the device 601 includes processing circuitry. The processing circuitry includes one or more of the elements discussed next with reference to FIG. 20. The device 701, may include other components not explicitly illustrated in FIG. 20 such as a CPU, GPU, frame buffer, etc. In FIG. 20, the device 701 includes a CPU 600 which performs the processes described above/below. The process data and instructions may be stored in memory 602. These processes and instructions may also be stored on a storage medium disk 604 such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the device 601 communicates, such as a server or computer.

Further, the claimed advancements may be provided as a utility application, background daemon, or component of an operating system, or combination thereof, executing in conjunction with CPU 600 and an operating system such as Microsoft Windows, UNIX, Solaris, LINUX, Apple MAC-OS and other systems known to those skilled in the art.

The hardware elements in order to achieve the device 601 may be realized by various circuitry elements, known to those skilled in the art. For example, CPU 600 may be a Xenon or Core processor from Intel of America or an Opteron processor from AMD of America, or may be other processor types that would be recognized by one of ordinary skill in the art. Alternatively, the CPU 600 may be implemented on an FPGA, ASIC, PLD or using discrete logic circuits, as one of ordinary skill in the art would recognize. Further, CPU 600 may be implemented as multiple processors cooperatively working in parallel to perform the instructions of the processes described above.

The device 601 in FIG. 20 also includes a network controller 606, such as an Intel Ethernet PRO network interface card from Intel Corporation of America, for interfacing with network 650, and to communicate with the other devices of FIG. 1. As can be appreciated, the network 650 can be a public network, such as the Internet, or a private network such as an LAN or WAN network, or any combination thereof and can also include PSTN or ISDN sub-networks. The network 650 can also be wired, such as an Ethernet network, or can be wireless such as a cellular network including EDGE, 3G, 4G and 5G wireless cellular systems. The wireless network can also be WiFi, Bluetooth, or any other wireless form of communication that is known.

The device 601 further includes a display controller 608, such as a NVIDIA Geforce GTX or Quadro graphics adaptor from NVIDIA Corporation of America for interfacing with display 610, such as an LCD monitor. A general purpose I/O interface 612 interfaces with a keyboard and/or mouse 614 as well as a touch screen panel 616 on or separate from display 610. General purpose I/O interface also connects to a variety of peripherals 618 including printers and scanners.

A sound controller 620 is also provided in the device 601 to interface with speakers/microphone 622 thereby providing sounds and/or music.

The general purpose storage controller 624 connects the storage medium disk 604 with communication bus 626, which may be an ISA, EISA, VESA, PCI, or similar, for interconnecting all of the components of the device 601. A description of the general features and functionality of the display 610, keyboard and/or mouse 614, as well as the display controller 608, storage controller 624, network controller 606, sound controller 620, and general purpose I/O interface 612 is omitted herein for brevity as these features are known.

Embodiments of the present disclosure may also be as set forth in the following parentheticals.

(1) An apparatus, comprising processing circuitry configured to obtain an image, apply a first model to the image, apply a second model to the image, determine, based on the first model, preliminary locations of objects in the obtained image, determine, based on the second model, adhesion locations disposed between the objects in the obtained image, and determine, based on the preliminary locations and the adhesion locations, refined locations of the objects in the obtained image

(2) The apparatus of (1), wherein the processing circuitry is further configured to before applying the first model and the second model to the obtained image, determine, using a trained neural network, a region of interest within which the objects are disposed and apply the first model and the second model to only the region of interest.

(3) The apparatus of (2), wherein the region of interest includes a jaw, the jaw includes an upper jaw and a lower jaw, and the processing circuitry is further configured to determine a first set of the refined locations corresponding to the upper jaw and a second set of the refined locations corresponding to the lower jaw based on a proximity of the objects to the upper jaw or the lower jaw.

(4) The apparatus of any one of (1) to (3), wherein the processing circuitry is further configured to generate the first model using a first trained neural network, and generate the second model using a second trained neural network.

(5) The apparatus of (4), wherein the second trained neural network is trained using a synthetically generated dataset of teeth having adhesion locations between the teeth.

(6) The apparatus of (4), wherein the first trained neural network is trained using 3D models mapped onto a teeth location mask using a voxelization process.

(7) The apparatus of any one of (1) to (6), wherein processing circuitry is further configured to determine, based on the second model, the adhesion locations disposed between the objects in the obtained image by determining the adhesion locations between objects disposed in an upper jaw and objects disposed in a lower jaw, and determining the adhesion locations between adjacent, neighboring objects in a same jaw.

(8) The apparatus of any one of (1) to (7), wherein the processing circuitry is further configured to determine the refined locations of the objects in the obtained image by applying a binary mask of the adhesion locations generated via the second model to a probability map of the objects generated via the first model to generate a probability map of predetermined possible locations of object adhesion, applying a predetermined threshold to the probability map of predetermined possible locations of object adhesion to decouple objects previously coupled as a single object due to the adhesion locations, and determining a minimum value of the threshold which decouples all of the objects and applying the determined minimum threshold value to the probability map of the predetermined possible locations of object adhesion.

(9) The apparatus of (8), wherein the processing circuitry is further configured to generate the binary mask of the adhesion locations by enlarging a mask applied to the objects in the obtained image by a predetermined amount to cause the mask around the objects to intersect with one another.

(10) The apparatus of (8), wherein the processing circuitry is further configured to generate a decoupled objects model based on the determined refined locations.

(11) A method, comprising obtaining an image; applying a first model to the image; applying a second model to the image; determining, based on the first model, preliminary locations of objects in the obtained image; determining, based on the second model, adhesion locations disposed between the objects in the obtained image; and determining, based on the preliminary locations and the adhesion locations, refined locations of the objects in the obtained image.

(12) The method of (11), further comprising before applying the first model and the second model to the obtained image, determining, using a trained neural network, a region of interest within which the objects are disposed and applying the first model and the second model to only the region of interest.

(13) The method of (12), wherein the region of interest includes a jaw, the jaw including an upper jaw and a lower jaw, and the method further comprises determining a first set of the refined locations corresponding to the upper jaw and a second set of the refined locations corresponding to the lower jaw based on a proximity of the objects to the upper jaw or the lower jaw.

(14) The method of any one of (11) to (13), further comprising generating the first model using a first trained neural network, and generating the second model using a second trained neural network.

(15) The method of (14), wherein the second trained neural network is trained using a synthetically generated dataset of teeth having adhesion locations between the teeth.

(16) The method of (14), wherein the first trained neural network is trained using 3D models mapped onto a teeth location mask using a voxelization process.

(17) The method of any one of (11) to (16), further comprising applying a classifier to remove noise from a dataset generated by applying the second model to the obtained image to determine the adhesion locations.

(18) The method of any one of (11) to (17), further comprising determining the refined locations of the objects in the obtained image by applying a binary mask of the adhesion locations generated via the second model to a probability map of the objects generated via the first model to generate a probability map of predetermined possible locations of object adhesion, applying a predetermined threshold to the probability map of predetermined possible locations of object adhesion to decouple objects previously coupled as a single object due to the adhesion locations, and determining a minimum value of the threshold which decouples all of the objects and applying the determined minimum threshold value to the probability map of the predetermined possible locations of object adhesion.

(19) The method of (18), wherein the binary mask of the adhesion locations is generated by enlarging a mask applied to the objects in the obtained image by a predetermined amount to cause the mask around the objects to intersect with one another.

(20) The method of (18), further comprising generating a decoupled objects model based on the determined refined locations.

(21) A non-transitory computer-readable storage medium storing computer-readable instructions that, when executed by a computer, cause the computer to perform a method of pixel-based classification of medical images, the method comprising: obtaining an image; applying a first model to the image; applying a second model to the image; determining, based on the first model, preliminary locations of objects in the obtained image; determining, based on the second model, adhesion locations disposed between the objects in the obtained image; and determining, based on the preliminary locations and the adhesion locations, refined locations of the objects in the obtained image.

(22) The method of (21), further comprising before applying the first model and the second model to the obtained image, determining, using a trained neural network, a region of interest within which the objects are disposed and applying the first model and the second model to only the region of interest.

(23) The method of (22), wherein the region of interest includes a jaw, the jaw including an upper jaw and a lower jaw, and the method further comprises determining a first set of the refined locations corresponding to the upper jaw and a second set of the refined locations corresponding to the lower jaw based on a proximity of the objects to the upper jaw or the lower jaw.

(24) The method of any one of (21) to (23), further comprising generating the first model using a first trained neural network, and generating the second model using a second trained neural network.

(25) The method of (24), wherein the second trained neural network is trained using a synthetically generated dataset of teeth having adhesion locations between the teeth.

(26) The method of (24), wherein the first trained neural network is trained using 3D models mapped onto a teeth location mask using a voxelization process.

(27) The method of any one of (21) to (26), further comprising applying a classifier to remove noise from a dataset generated by applying the second model to the obtained image to determine the adhesion locations.

(28) The method of any one of (21) to (27), further comprising determining the refined locations of the objects in the obtained image by applying a binary mask of the adhesion locations generated via the second model to a probability map of the objects generated via the first model to generate a probability map of predetermined possible locations of object adhesion, applying a predetermined threshold to the probability map of predetermined possible locations of object adhesion to decouple objects previously coupled as a single object due to the adhesion locations, and determining a minimum value of the threshold which decouples all of the objects and applying the determined minimum threshold value to the probability map of the predetermined possible locations of object adhesion.

(29) The method of (28), wherein the mask of the adhesion locations is generated by enlarging a mask applied to the objects in the obtained image by a predetermined amount to cause the mask around the objects to intersect with one another.

(30) The method of (28), further comprising generating a decoupled objects model based on the determined refined locations.

Obviously, numerous modifications and variations are possible in light of the above teachings. It is therefore to be understood that within the scope of the appended claims, the invention may be practiced otherwise than as specifically described herein.

Thus, the foregoing discussion discloses and describes merely exemplary embodiments of the present invention. As will be understood by those skilled in the art, the present invention may be embodied in other specific forms without departing from the spirit or essential characteristics thereof. Numerous modification and variations on the present invention are possible in light of the above teachings. Accordingly, the disclosure of the present invention is intended to be illustrative, but not limiting of the scope of the invention, as well as other claims. The disclosure, including any readily discernible variants of the teachings herein, defines, in part, the scope of the foregoing claim terminology such that no inventive subject matter is dedicated to the public.

All publications, patent applications, patents, and other references mentioned herein are incorporated by reference in their entirety. Further, the materials, methods, and examples are illustrative only and are not intended to be limiting, unless otherwise specified.

1. An apparatus, comprising:
processing circuitry configured to
obtain an image,
apply a first model to the image,
apply a second model to the image,
determine, based on the first model, preliminary locations of objects in the obtained image,
determine, based on the second model, adhesion locations disposed between the objects in the obtained image, and
determine, based on the preliminary locations and the adhesion locations, refined locations of the objects in the obtained image.

2. The apparatus of claim 1, wherein the processing circuitry is further configured to
before applying the first model and the second model to the obtained image, determine, using a trained neural network, a region of interest within which the objects are disposed, and
apply the first model and the second model to only the region of interest.

3. The apparatus of claim 2, wherein
the region of interest includes a jaw,
the jaw includes an upper jaw and a lower jaw, and
the processing circuitry is further configured to determine a first set of the refined locations corresponding to the upper jaw and a second set of the refined locations corresponding to the lower jaw based on a proximity of the objects to the upper jaw or the lower jaw.

4. The apparatus of claim 1, wherein the processing circuitry is further configured to
generate the first model using a first trained neural network, and
generate the second model using a second trained neural network.

5. The apparatus of claim 4, wherein the second trained neural network is trained using a synthetically generated dataset of teeth having adhesion locations between the teeth.

6. The apparatus of claim 4, wherein the first trained neural network is trained using 3D models mapped onto a teeth location mask using a voxelization process.

7. The apparatus of claim 1, wherein processing circuitry is further configured to determine, based on the second model, the adhesion locations disposed between the objects in the obtained image by
determining the adhesion locations between objects disposed in an upper jaw and objects disposed in a lower jaw, and
determining the adhesion locations between adjacent, neighboring objects in a same jaw.

8. The apparatus of claim 1, wherein the processing circuitry is further configured to determine the refined locations of the objects in the obtained image by
applying a binary mask of the adhesion locations generated via the second model to a probability map of the objects generated via the first model to generate a probability map of predetermined possible locations of object adhesion,
applying a predetermined threshold to the probability map of predetermined possible locations of object adhesion to decouple objects previously coupled as a single object due to the adhesion locations, and
determining a minimum value of the threshold which decouples all of the objects and applying the determined minimum threshold value to the probability map of the predetermined possible locations of object adhesion.

9. The apparatus of claim 8, wherein the processing circuitry is further configured to generate the binary mask of the adhesion locations by enlarging a mask applied to the objects in the obtained image by a predetermined amount to cause the mask around the objects to intersect with one another.

10. The apparatus of claim 8, wherein the processing circuitry is further configured to generate a decoupled objects model based on the determined refined locations.

11. A method, comprising:
obtaining an image;
applying a first model to the image;
applying a second model to the image;
determining, based on the first model, preliminary locations of objects in the obtained image;
determining, based on the second model, adhesion locations disposed between the objects in the obtained image; and
determining, based on the preliminary locations and the adhesion locations, refined locations of the objects in the obtained image.

12. The method of claim 11, further comprising
before applying the first model and the second model to the obtained image, determining, using a trained neural network, a region of interest within which the objects are disposed and applying the first model and the second model to only the region of interest.

13. The method of claim 12, wherein
the region of interest includes a jaw, the jaw including an upper jaw and a lower jaw, and
the method further comprises determining a first set of the refined locations corresponding to the upper jaw and a second set of the refined locations corresponding to the lower jaw based on a proximity of the objects to the upper jaw or the lower jaw.

14. The method of claim 11, further comprising
generating the first model using a first trained neural network, and
generating the second model using a second trained neural network.

15. The method of claim 14, wherein the second trained neural network is trained using a synthetically generated dataset of teeth having adhesion locations between the teeth.

16. The method of claim 14, wherein the first trained neural network is trained using 3D models mapped onto a teeth location mask using a voxelization process.

17. The method of claim 11, further comprising applying a classifier to remove noise from a dataset generated by applying the second model to the obtained image to determine the adhesion locations.

18. The method of claim 11, further comprising determining the refined locations of the objects in the obtained image by
applying a binary mask of the adhesion locations generated via the second model to a probability map of the objects generated via the first model to generate a probability map of predetermined possible locations of object adhesion,
applying a predetermined threshold to the probability map of predetermined possible locations of object adhesion to decouple objects previously coupled as a single object due to the adhesion locations, and
determining a minimum value of the threshold which decouples all of the objects and applying the determined minimum threshold value to the probability map of the predetermined possible locations of object adhesion.

19. The method of claim 18, wherein the binary mask of the adhesion locations is generated by enlarging a mask applied to the objects in the obtained image by a predetermined amount to cause the mask around the objects to intersect with one another.

20. A non-transitory computer-readable storage medium storing computer-readable instructions that, when executed by a computer, cause the computer to perform a method of pixel-based classification of medical images, the method comprising:
obtaining an image;
applying a first model to the image;
applying a second model to the image;
determining, based on the first model, preliminary locations of objects in the obtained image;
determining, based on the second model, adhesion locations disposed between the objects in the obtained image; and
determining, based on the preliminary locations and the adhesion locations, refined locations of the objects in the obtained image.
